(19) United States
(12) Patent Application Publication
(10) Pub. No.: US 2023/0215570 A1
(43) Pub. Date: Jul. 6, 2023

(54) METHOD AND APPARATUS FOR DIAGNOSING COLON PLYP USING MACHINE LEARNING MODEL

(71) Applicant: HEM PHARMA INC., Pohang-si (KR)

(72) Inventors: Yo Sep JI, Suwon-si (KR); So Young PARK, Suwon-si (KR)

(21) Appl. No.: 18/181,387

(22) Filed: Mar. 9, 2023

Related U.S. Application Data
(63) Continuation of application No. PCT/KR2021/012253, filed on Sep. 9, 2021.

(30) Foreign Application Priority Data
Oct. 20, 2020 (KR) 10-2020-0136235

(51) Int. Cl. G16H 50/20; C12Q 1/04

(52) U.S. Cl. CPC G16H 50/20; C12Q 1/045

(57) ABSTRACT

A method of diagnosing the presence or absence of colon polyps by using a machine learning model, which is performed by a diagnostic apparatus, includes: analyzing a mixture of a sample collected from a subject and a gut environment-like composition; extracting a plurality of microbial data based on an analysis result of the mixture; selecting a microbe-related feature to be used for the machine learning model from the plurality of microbial data based on a predetermined feature selection algorithm; training the machine learning model by using the microbe-related feature to predict the presence or absence of colon polyps for each of the microbial data; and diagnosing the presence or absence of colon polyps based on an output value of the machine learning model by inputting, into the trained machine learning model, the microbial data extracted based on the analysis result of the mixture of the sample collected from the subject and the gut environment-like composition, wherein the microbe-related feature includes the content of at least one kind of microbes selected from families belonging to the order Oscillospirales, the order Burkholderiales, the order Saccharimonadales, the order Lactobacillales, the order Bacteroidales, the order Clostridiales, the order Erysipelotrichales, the order Bacteroidales and the order Lachnospirales.

### TECHNICAL FIELD

The present disclosure relates to method and apparatus for diagnosing colon plyp using machine learning model.

### BACKGROUND

Colorectal cancer is a malignant tumor composed of cancer cells generated in the colon, and is the third most common cancer type worldwide. Also, it is known that more than 1 million cases occur annually. Colorectal cancer has a 5-year survival rate of 90% when diagnosed in its early stages. In most cases, colorectal cancer has no symptoms in its early stages, but is discovered only after it has progressed to stage 3 or 4. Therefore, it is known that metastasis is the major cause of death in patients with colorectal cancer.

Colon cancer can be diagnosed based on a biopsy sample obtained during colonoscopy. However, since colorectal cancer generally has no symptoms in its early stages, its diagnosis is quite difficult.

Meanwhile, the term “genome” refers to genes contained in chromosomes, the term “microbiota” refers to a collection of microbes found in a specific environment, and the “microbiome” refers to genes in all the collection of microbes in the environment. Herein, the tern “microbiome” may refer to a combination of genome and microbiota.

Recently, there has been an attempt to diagnose colon cancer by identifying microbes that can act as causative factors of colorectal cancer through metagenome analysis of microbiota.

In this regard, Korean Patent No. 10-2057047, which is the prior art, relates to a disease prediction apparatus and a disease prediction method using the same, and discloses a disease prediction method for predicting a disease of a predetermined person by comparing a learning vector with a predetermined person vector extracted from a biosignal of the predetermined person.

However, according to the prior art, bacterial metagenome analysis is performed without a special process such as culturing of samples, and, thus, it is difficult to accurately find the causative factor of colorectal cancer due to a large bias between samples of respective subjects.

Also, when a machine learning model is trained using unprocessed samples of respective subjects as training data, the training data may have a lot of noise, and, thus, the performance of the machine learning model may be significantly degraded.

### DISCLOSURE OF THE INVENTION

### Problems to Be Solved by the Invention

The present disclosure is to solve the above problems, and is to improve the performance of a machine learning model for diagnosing the presence or absence of colon polyps by selecting microbe-related features from a plurality of microbial data based on an analysis result of a mixture of a sample and a gut environment-like composition.

However, the problems to be solved by this disclosure are not limited to those mentioned above, and other problems not mentioned will be clearly understood by those skilled in the art from the following description. means for solving the problems

To solve the problems, one example of the present disclosure provides a method of diagnosing the presence or absence of colon polyps by using a machine learning model, which is performed by a diagnostic apparatus, comprising: a process of analyzing a mixture of a sample collected from a subject and a gut environment-like composition; a process of extracting a plurality of microbial data based on an analysis result of the mixture; a process of selecting a microbe-related feature to be used for the machine learning model from the plurality of microbial data based on a predetermined feature selection algorithm; a process of training the machine learning model by using the microbe-related feature to predict the presence or absence of colon polyps for each of the microbial data; and a process of diagnosing the presence or absence of colon polyps based on an output value of the machine learning model by inputting, into the trained machine learning model, the microbial data extracted based on the analysis result of the mixture of the sample collected from the subject and the gut environment-like composition, wherein the microbe-related feature includes the content of at least one kind of microbes selected from families belonging the order Oscillospirales, the order Burkholderiales, the order Saccharimonadales, the order Lactobacillales, the order Bacteroidales, the order Clostridiales, the order Erysipelotrichales, the order Bacteroidales and the order Lachnospirales.

Also, another example of the present disclosure provides an apparatus of diagnosing the presence or absence of colon polyps by using a machine learning model, comprising: a microbial data extraction unit that extracts a plurality of microbial data based on an analysis result of a mixture of a gut-derived substance collected from a subject and a gut environment-like composition; a feature selection unit that selects a microbe-related feature to be used for the machine learning model from the plurality of microbial data based on a predetermined feature selection algorithm; a training unit that trains the machine learning model by using the microbe-related feature to predict the presence or absence of colon polyps for each of the microbial data; and a diagnosis unit that diagnoses colon polyps based on the presence or absence of colon polyps, which is an output value of the machine learning model, by inputting, into the trained machine learning model, the microbial data extracted based on the analysis result of the mixture of the gut-derived substance collected from the subject and the gut environment-like composition, wherein the microbe-related feature includes the content of at least one kind of microbes selected from the family Oscillospiraceae, the family Streptococcaceae, the family Enterococcaceae, the family Marinifilaceae, the family Lactobacillaceae, the family Clostridiaceae, the family Leuconostocaceae, the family Erysipelatoclostridiaceae and the family Lachnospiraceae.

The above-described problem solving means are merely illustrative and should not be construed as intended to limit the present invention. In addition to the above-described exemplary embodiments, there may be additional embodiments described in the drawings and detailed descriptions of the invention.

### Effects of the Invention

According to any one of the above-described means for solving the problems of the present disclosure, it is possible to improve the performance of a machine learning model for diagnosing the presence or absence of colon polyps by selecting microbe-related features from a plurality of microbial data based on an analysis result of a mixture of a gut-derived substance and a gut environment-like composition.

### BRIEF DESCRIPTION OF THE DRAWINGS

FIG. 1 is a block diagram illustrating a diagnostic apparatus according to an example of the present disclosure.

FIG. 2 is a diagram illustrating an MCMOD technique according to an example of the present disclosure.

FIG. 3 is a diagram for explaining a sample analysis through the MCMOD technique according to an example of the present disclosure.

FIG. 4 is a diagram for explaining the interpretation of a sample analysis result through the MCMOD technique according to an example of the present disclosure.

FIGS. 5A-5C are diagrams for explaining selected microbe-related features according to an example of the present disclosure.

FIGS. 6A-6C are diagrams comparing analysis results of respective samples according to a method of diagnosing the presence or absence of colon polyps of an example of the present disclosure and a method of Comparative Example.

FIGS. 7A-7B are diagrams comparing analysis results of respective samples according to the method of diagnosing the presence or absence of colon polyps of an example of the present disclosure and the method of Comparative Example.

FIGS. 8A-8B are diagrams comparing machine learning models in performance according to the method of diagnosing the presence or absence of colon polyps of an example of the present disclosure and the method of Comparative Example.

FIG. 9 is a diagram illustrating changes in performance of machine learning models depending on features according to the method of diagnosing the presence or absence of colon polyps of an example of the present disclosure and the method of Comparative Example.

FIGS. 10A-10B are diagrams comparing random forest models in performance according to the method of diagnosing the presence or absence of colon polyps of an example of the present disclosure and the method of Comparative Example.

FIGS. 11A-11B are diagrams comparing XGB models in performance according to the method of diagnosing the presence or absence of colon polyps of an example of the present disclosure and the method of Comparative Example.

FIG. 12 is a flowchart illustrating a method of diagnosing the presence or absence of colon polyps according to an example of the present disclosure.

### BEST MODE FOR CARRYING OUT THE INVENTION

A Hereafter, embodiments of the present disclosure will be described in detail with reference to the accompanying drawings so that the present disclosure may be readily implemented by a person with ordinary skill in the art. However, it is to be noted that the present disclosure is not limited to the embodiments but may be embodied in various other ways. In drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected” another element and an element being “electronically connected” to another element via another element. Further, it is to be understood that the terms “comprises,” “includes,” “comprising,” and/or “including” means that one or more other components, steps, operations, and/or elements are not excluded from the described and recited systems, devices, apparatuses, and methods unless context dictates otherwise; and is not intended to preclude the possibility that one or more other components, steps, operations, parts, or combinations thereof may exist or may be added.

Throughout the whole document, the term “unit” includes a unit implemented by hardware or software and a unit implemented by both of them. One unit may be implemented by two or more pieces of hardware, and two or more units may be implemented by one piece of hardware.

In the present specification, some of operations or functions described as being performed by a device may be performed by a server connected to the device. Likewise, some of operations or functions described as being performed by a server may be performed by a device connected to the server.

Hereinafter, embodiments of the present disclosure will be described in detail with reference to the accompanying drawings.

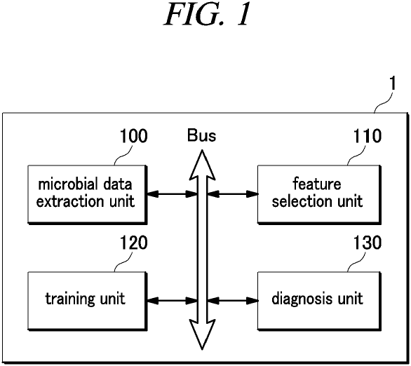

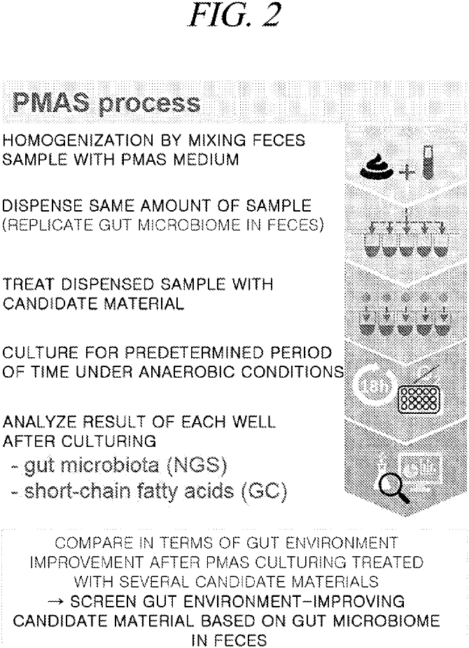

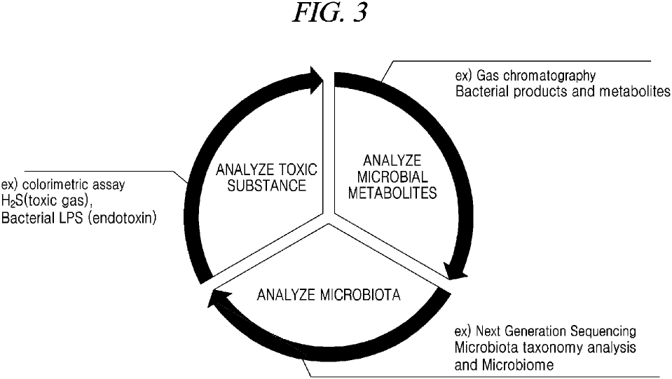

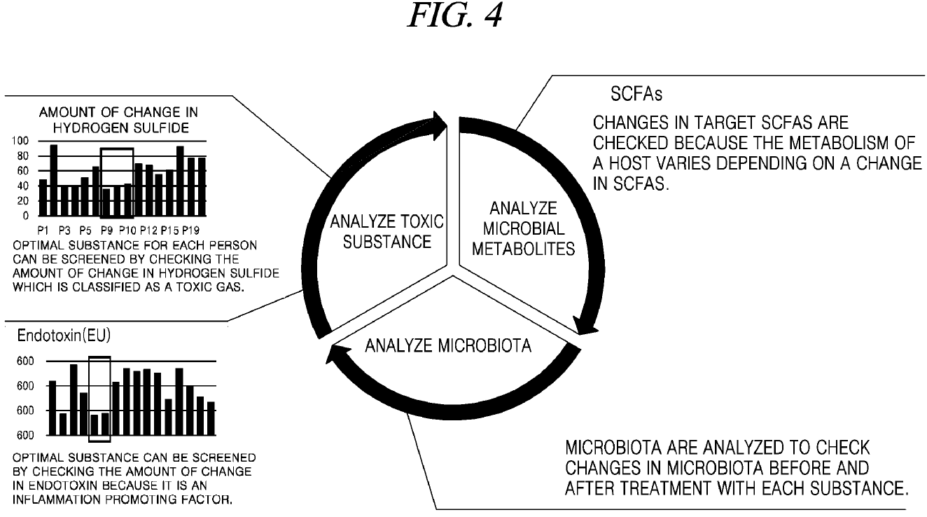

FIG. 1 is a block diagram illustrating a diagnostic apparatus according to an example of the present disclosure. Referring to FIG. 1, a diagnostic apparatus 1 may include a microbial data extraction unit 100, a feature selection unit 110, a training unit 120, and a diagnosis unit 130.

Examples of the diagnostic apparatus 1 may include a personal computer such as a desktop computer or a laptop computer, as well as a mobile device capable of wired/wireless communication. The mobile device is a wireless communication device that ensures portability and mobility and may include a smartphone, a tablet PC, a wearable device and various kinds of devices equipped with a communication module such as Bluetooth (BLE, Bluetooth Low Energy), NFC, RFID, ultrasonic waves, infrared rays, WiFi, LiFi, and the like. However, the diagnostic apparatus 1 is not limited to the shape illustrated in FIG. 1 or the above examples.

The diagnostic apparatus 1 may detect a biomarker for diagnosing the presence or absence of colon polyps caused by abnormalities in the gut environment in a sample collected from a subject.

For example, the diagnostic apparatus 1 may diagnose the presence or absence of colon polyps based on a sample preparation process, a sample pretreatment process, a sample analysis process, a data analysis process, and derived data.

In an embodiment, the biomarker may be a substance detected in the gut, and specifically, it may include microbiota, endotoxins, hydrogen sulfide, gut microbial metabolites, short-chain fatty acids and the like, but is not limited thereto.

The microbial data extraction unit 100 may extract a plurality of microbial data based on an analysis result of a mixture of a sample collected from a subject and a gut environment-like composition. Herein, the plurality of microbial data may be classified into a training set to be used for training and a test set, and a classification ratio may vary, such as 9:1, 7:3, 5:5 and the like, and may be preferably 7:3.

According to the present disclosure, pretreatment for analyzing a mixture of a sample and a gut environment-like composition is performed. In the present disclosure, the pretreatment may be referred to as MCMOD (Meta-culture Multi-Omics Diagnose).

For example, an in-vitro analysis of fecal microbiome and metabolites is performed to feces samples obtained from humans and various animals that can most easily represent the gut microbial environment in vivo.

Herein, the term “subject” refers to any living organism which may have a gut disorder, may have a disease caused by a gut disorder or develop it or may be in need of an improvement of gut environment. Specific examples thereof may include, but not limited to, mammals such as mice, monkeys, cattle, pigs, minipigs, domestic animals and humans, birds, cultured fish, and the like.

The term “sample” refers to a material derived from the subject and specifically may be cells, urine, feces, or the like, but may not be limited thereto as long as a material, such as microbiota, gut microbial metabolites, endotoxins and short-chain fatty acids, present in the gut can be detected therefrom.

The term “gut environment-like composition” may refer to a composition prepared for mimicking identically/similarly mimicking the gut environment of the subject in vitro. For example, the gut environment-like composition may be a culture medium composition, but is not limited thereto.

The gut environment-like composition may include L-cysteine hydrochloride and mucin.

Herein, the term “L-cysteine hydrochloride” is one of amino acid supplements and plays an important role in metabolism as a component of glutathione in vivo and is also used to inhibit browning of fruit juices and oxidation of vitamin C.

L-cysteine hydrochloride may be contained at a concentration of, for example, from 0.001% (w/v) to 5% (w/v), specifically from 0.01% (w/v) to 0.1% (w/v).

L-cysteine hydrochloride is one of various formulations or forms of L-cysteine, and the composition may include L-cysteine including other types of salts as well as L-cysteine.

The term “mucin” is a mucosubstance secreted by the mucous membrane and includes submandibular gland mucin and others such as gastric mucosal mucin and small intestine mucin. Mucin is one of glycoproteins and known as one of energy sources such as carbon sources and nitrogen sources that gut microbiota can actually use.

Mucin may be contained at a concentration of, for example, 0.01% (w/v) to 5% (w/v), specifically, from 0.1% (w/v) to 1% (w/v), but is not limited thereto.

In an embodiment, the gut environment-like composition may not include any nutrient other than mucin and specifically may not include a nitrogen source and/or carbon source such as protein and carbohydrate.

The protein that serves as a carbon source and nitrogen source may include one or more of tryptone, peptone and yeast extract, but may not be limited thereto. Specifically, the protein may be tryptone.

The carbohydrate that serves as a carbon source may include one or more of monosaccharides such as glucose, fructose and galactose and disaccharides such as maltose and lactose, but may not be limited thereto. Specifically, the carbohydrate may be glucose.

In an embodiment, the gut environment-like composition may not include glucose and tryptone, but is not limited thereto.

The gut environment-like composition may further include one or more selected from the group consisting of sodium chloride (NaCl), sodium carbonate (NaHCO₃), potassium chloride (KCl) and hemin. Specifically, sodium chloride may be contained at a concentration of, for example, from 10 mM to 100 mM, sodium carbonate may be contained at a concentration of, for example, from 10 mM to 100 mM, potassium chloride may be contained at a concentration of, for example, from 1 mM to 30 mM, and hemin may be contained at a concentration of, for example, from 1 × 10⁻⁶ g/L to 1 × 10⁻⁴ g/L, but is not limited thereto.

In the pretreatment, the mixture may be cultured for 18 to 24 hours under anaerobic conditions.

For example, in an anaerobic chamber, the same amount of a homogenized feces-medium mixture is dispensed to each of culture plates such as 96-well plates. Herein, the culture may be performed for 12 hours to 48 hours, specifically, for 18 hours to 24 hours, but is not limited thereto.

Then, the plates are cultured under anaerobic conditions with temperature, humidity and motion similar to those of the gut environment to ferment and culture the respective test groups.

After the culturing of the mixture, a culture in which the mixture has been cultured is analyzed. The analysis of the culture may be to extract microbial data including at least one of the content, concentration and kind of one or more of endotoxins, hydrogen sulfides, short-chain fatty acids (SCFAs) and microbiota-derived metabolites contained in the culture, and a change in kind, concentration, content or diversity of bacteria included in the microbiota, but is not limited thereto.

Herein, the term “endotoxin” is a toxic substance that can be found inside a bacterial cell and acts as an antigen composed of a complex of proteins, polysaccharides, and lipids. In an embodiment, the endotoxin may include lipopolysaccharides (LPS), but may not limited thereto, and the LPS may be specifically gram negative and pro-inflammatory.

The term “short-chain fatty acid (SCFA)” refers to a short-length fatty acid with six or fewer carbon atoms and is a representative metabolite produced from gut microbiota. The SCFA has useful functions in the body, such as an increase in immunity, stabilization of gut lymphocytes, a decrease in insulin signaling, and stimulation of sympathetic nerves.

In an embodiment, the short-chain fatty acids may include one or more selected from the group consisting of formate, acetate, propionate, butyrate, isobutyrate, valerate and isovalerate, but may not be limited thereto.

The culture may be analyzed by various analysis methods, such as genetic analysis methods including absorbance analysis, chromatography analysis and next generation sequencing, and metagenomic analysis methods, that can be used by a person with ordinary skill in the art.

When the culture is analyzed, the culture may be centrifuged to separate a supernatant and a precipitate and then, the supernatant and the precipitate (pallet) may be analyzed. For example, metabolites, short-chain fatty acids, toxic substances, etc. from the supernatant and microbiota from the pallet may be analyzed.

For example, after the culturing is completed, toxic substances, such as hydrogen sulfide and bacterial LPS (endotoxin), microbial metabolites, such as short-chain fatty acids, from the supernatant obtained by centrifugation of the cultured test groups are analyzed through absorbance analysis and chromatography analysis, and a culture-independent analysis method is performed to the microbiota from the centrifuged pellet. For example, the amount of change in hydrogen sulfide produced by the culturing may be measured through a methylene blue method using N,N-dimethyl-p-phenylene-diamine and iron chloride (FeCl₃) and the level of endotoxins that is one of inflammation promoting factors may be measured using an endotoxin assay kit. Also, microbial metabolites such as short-chain fatty acids including acetate, propionate and butyrate can be analyzed through gas chromatography.

Microbiota can be analyzed by genome-based analysis through metagenomic analysis such as real-time PCR in which all genomes are extracted from a sample and a bacteria-specific primer suggested in the GULDA method or next generation sequencing.

According to the present disclosure, the culture is analyzed in a state where the gut environment is implemented in vitro by using the gut environment-like composition, and, thus, it is possible to reduce a bias between training data by optimizing the training data before machine learning.

Accordingly, it is possible to facilitate selection of microbe-related features to be described later and also improve the performance of a machine learning model by training the machine learning model based on the microbe-related features. Therefore, it is possible to increase the accuracy in diagnosing the presence or absence of colon polyps through the trained machine learning model.

The feature selection unit 110 may perform selection (i.e., feature selection) of microbe-related features from a plurality of microbial data as features to be used for the machine learning model based on a predetermined feature selection algorithm. The number of the microbe-related features may be 6 to 16. For example, the number of the microbe-related features may be 16.

Features (, variables or attributes) are used in creating a machine learning model. If a large number of features or inappropriate features are used, the machine learning model may overfit data or the prediction accuracy may decrease.

Accordingly, in order for the machine learning model to have a high prediction accuracy, it is necessary to use an appropriate combination of features. That is, it is possible to reduce the complexity of the machine learning model while using as few features as possible by selecting features most closely related to a response feature to be predicted.

The feature selection algorithm may include at least one of, for example, a Boruta algorithm and a recursive feature elimination (RFE) algorithm.

The microbe-related features selected from the predetermined feature selection algorithm may include the content of at least one kind of microbes selected from families belonging to the order Oscillospirales, the order Burkholderiales, the order Saccharimonadales, the order Lactobacillales, the order Bacteroidales, the order Clostridiales, the order Erysipelotrichales, the order Bacteroidales and the order Lachnospirales.

In an embodiment, the microbe-related features selected from the predetermined feature selection algorithm may further include the content of at least one kind of microbes selected from genera belonging to, for example, the family Oscillospiraceae, the family Streptococcaceae, the family Enterococcaceae, the family Marinifilaceae, the family Lactobacillaceae, the family Clostridiaceae, the family Leuconostocaceae, the family Erysipelatoclostridiaceae and the family Lachnospiraceae.

In an embodiment, the microbe-related features selected from the predetermined feature selection algorithm may further include the content of at least one kind of microbes selected from species belonging to genera, for example, the genus Enterococcus, the genus Odoribacter, the genus Streptococcus, the genus Lactobacillus, the genus Clostridium sensu stricto, the genus leuconostoc, the genus Erysipelatoclostridium and the genus Eisenbergiella.

The training unit 120 may train the machine learning model using the microbe-related features.

For example, the training unit 120 may perform supervised learning based on labeling of the presence or absence of colon polyps for each of microbial data (training data) and the content of microbes related to the selected feature so that the machine learning model can be trained to predict the presence or absence of colon polyps for each of microbial data.

The machine learning model may include at least one of, for example, a logistic regression model, a glmnet model, a random forest model, a gradient boosting model and an extreme gradient boost (XGB) model.

The diagnosis unit 130 may input the extracted microbial data into the trained machine learning model based on an analysis result of a mixture of a gut-derived substance collected from a test subject and the gut environment-like composition to diagnose the presence or absence of colon polyps.

For example, the diagnosis unit 130 may diagnose colon polyps based on the presence or absence of colon polyps which is an output value of the machine learning model.

Hereinafter, embodiments of the present disclosure will be described in detail. However, the present disclosure is not limited thereto.

### EXAMPLES

### Example 1. Microbe-Related Features Selected Based on Recursive Feature Elimination Algorithm After MCMOD

The following test was performed in order to check microbe-related features selected based on the recursive feature elimination algorithm after MCMOD of Example 1.Feces collected from 77 colon polyp patients and 61 normal people were used as respective samples, as shown in Table 1 below.

TABLE 1

| Disease and Examination Item | Classification | Data Source (Collection Route) | Criteria for Disease | Number of Samples from Original Data | | | Original Data | | | | | |
|---|---|---|---|---|---|---|---|---|---|---|---|---|
| | | | | | | | Train Set | | | Test Set | | |
| | | | | Disease Group | Normal Group | Total | Disease Group | Normal Group | Total | Disease Group | Normal Group | Total |
| Colon Polyp | Test Result Sheet | Gibbeum Hospital | Medical Opinion | 77 | 61 | 138 | 61 | 43 | 104 | 16 | 18 | 34 |

The feces were treated with MCMOD to extract microbial data for each sample. The microbial data were classified into training data (training set) to be used for training and test data (test set) at a ratio of 7:3.

Thereafter, feature selection was performed on the training data through a recursive feature elimination algorithm to select microbe-related features to be used for the machine learning model. Meanwhile, the test data were used to evaluate the performance of the machine learning model, as will be described below.

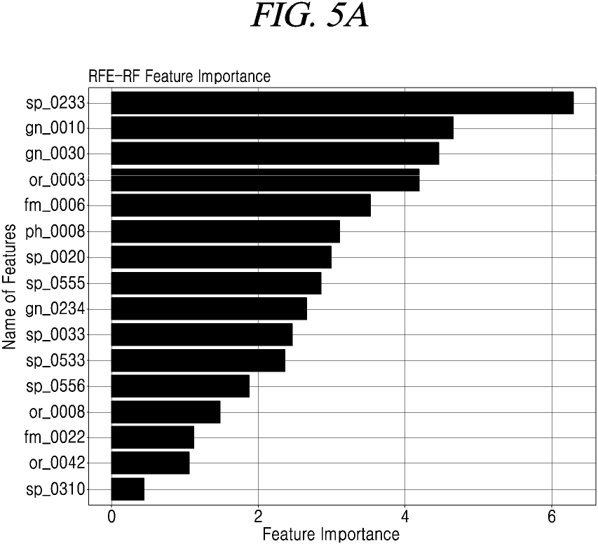

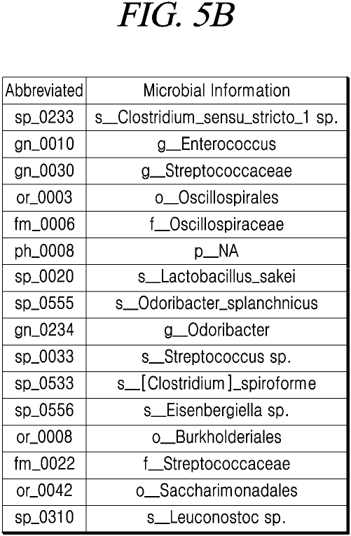

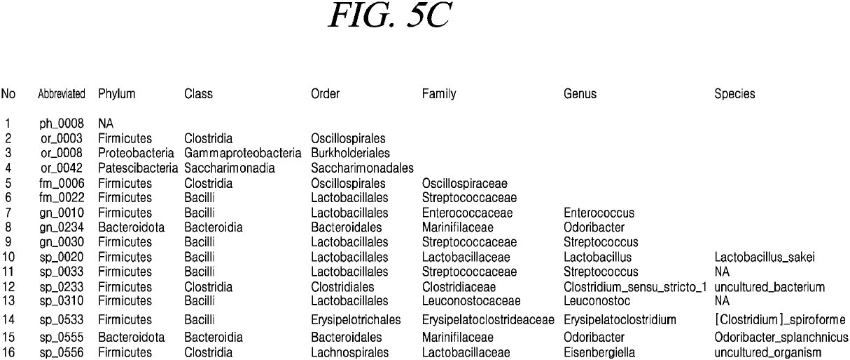

FIGS. 5A-5C are diagrams for explaining selected microbe-related features according to an example of the present disclosure.

Through the recursive feature elimination algorithm, 16 microbe-related features were selected as the feature group with the highest accuracy. FIG. 5A shows the importance (accuracy) of the selected microbe-related features, and FIG. 5B shows the selected microbe-related features.

Also, FIG. 5C shows taxonomic information of the selected microbe-related features.

In FIG. 5B and FIG. 5C, an alphabetic letter before the abbreviated name represents a taxonomic location. That is, “p” is Phylum, “c” is Class, “o” is Order, “f” is Family, “g” is Genus, and “s” is Species.

### Comparative Example 1. Analysis Results of Feces Samples Treated With MCMOD and Feces Samples Not Treated with MCMOD

Feces were collected from one subject for 8 days, and 8 feces samples (J01, J02, J03, J04, J06, J08, J09 and J10) sorted by date were treated with MCMOD and then subjected to next-generation sequencing to analyze genes of microbes (Example). Similarly, feces samples not treated with MCMOD were subjected to next-generation sequencing to analyze genes of microbes (Comparative Example).

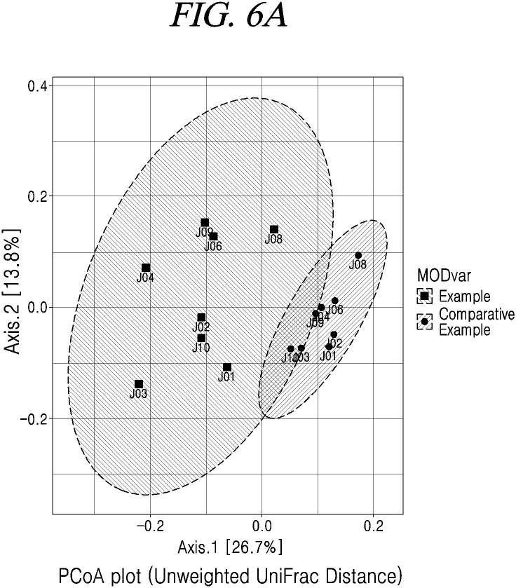

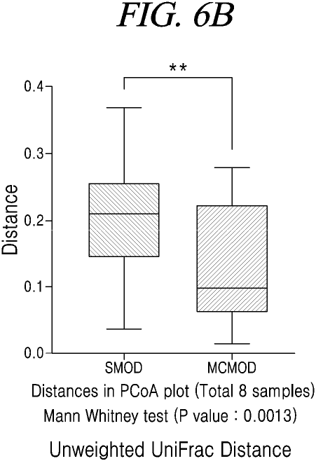

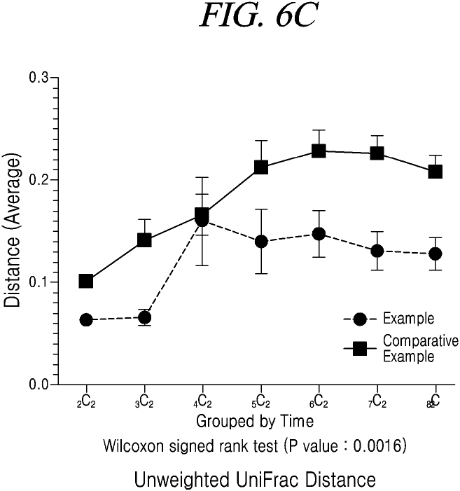

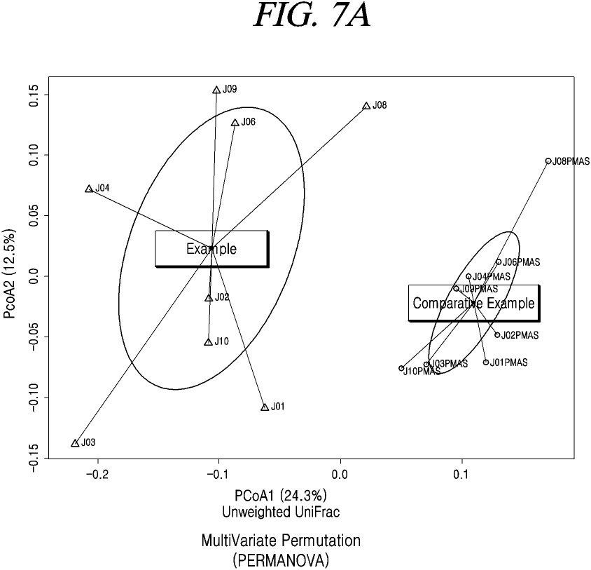

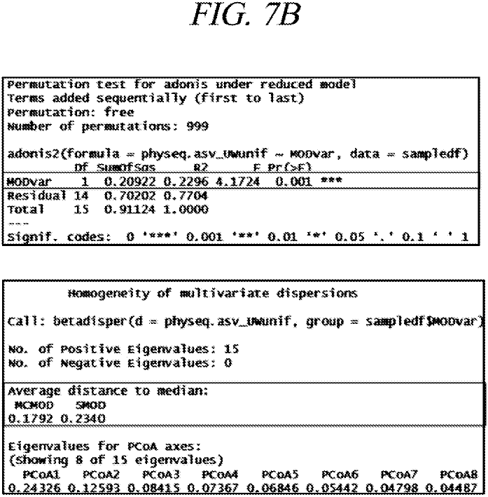

FIGS. 6A-6C are diagrams comparing analysis results of respective samples according to a method of diagnosing the presence or absence of colon polyps of an example of the present disclosure and a method of Comparative Example, and FIGS. 7A-7B are diagrams comparing analysis results of respective samples according to the method of diagnosing the presence or absence of colon polyps of an example of the present disclosure and the method of Comparative Example.

FIG. 6A shows, as a PCoA plot, the beta diversity of the feces sample by using the Unweighted Unifrac Distance. As shown in the PCoA plot of FIG. 6A, it can be seen that the feces samples treated with MCMOD are relatively clustered, whereas the feces samples not treated with MCMOD are relatively scattered.

FIG. 6B shows, as a box plot, the distances among 8 points in each group (Example and Comparative Example) on the PCoA plot.

As can be seen from the box plot, the differences among the feces samples of Example are statistically significantly smaller than those of Comparative Example.

FIG. 6C shows the distances among 8 points in each group (Example and Comparative Example) on the PCoA plot.

Since there are 8 samples in each group, each group has a total of 28 types of distances between two samples. The samples with 28 types of distances were grouped in chronological order from 2C2 to 8C2 .

Since a feces sample J01 was collected first and a feces sample J10 was collected last, the distance between the two samples collected first and second in the group C2C (N=1) (the distance between the samples J01 and J02) was calculated.

In the group 3C2 (N=3), the distances among the three samples including the next collected feces sample J03 (between J01 and J02, between J01 and J03, and between J02 and J03) were calculated to find the average and standard error of the distances.

In the group 4C2 (N=6), the distances among the four samples including the next collected feces sample J04 (between J01 and J02, between J01 and J03, between J01 and J04, between J02 and J03, between J02 and J04, and between J03 and J04) were calculated to find the average and standard error of the distances.

Similarly, in the group 8C2 (N=28), the distances among the eight samples including the last collected feces sample J10 (a total of 28 types of distances) were calculated to find the average and standard error of the distances.

As can be seen from the distance values in the PCoA plot, the differences among the feces sample groups (2C2 to 8C2) of Example are statistically significantly smaller than those of Comparative Example.

FIGS. 7A-7B show analysis results of the two groups (Example and Comparative Example) through PERMANOVA tests.

Based on the result of PERMANOVA tests as shown in FIG. 7B, a Pr(>F) value is as small as 0.001, which indicates that the two groups (Example and Comparative Example) are different in terms of population mean. This means there is a statistically significant difference between the two groups.

Also, it can be seen that the average distance to median of each feces sample in each group is smaller in Example (0.1792) than in Comparative Example (0.2340), which means that Example has less noise than Comparative Example.

As described above, the feces samples treated with MCMOD have relatively little noise due to a small bias between the feces samples and thus have low fluctuations.

That is, according to the present disclosure, the feces samples are treated with MCMOD before feature selection and machine learning training to facilitate feature selection, and, as will be described later, the machine learning model is trained to improve the performance of the machine learning model.

### Comparative Example 2. Comparison of Performance of Machine Learning Models Trained Using Training Data Obtained from Feces Sample Treated with MCMOD and Feces Sample Not Treated with MCMOD

The feces samples collected in Example 1 were treated with MCMOD to extract microbial data (Example), and microbial data were extracted without MCMOD treatment (Comparative Example).

Through the recursive feature elimination algorithm, 16 microbe-related features were selected from the microbial data in Example and 4 microbe-related features were selected from the microbial data in Comparative Example.

By using the microbial data and microbe-related features of Example and Comparative Example, a logistic regression analysis (LRA) model, a random forest (RF) model, a glmnet model, a gradient boosting model and an extreme gradient boost (XGB) model were trained. Then, the performance of each machine learning model was evaluated.

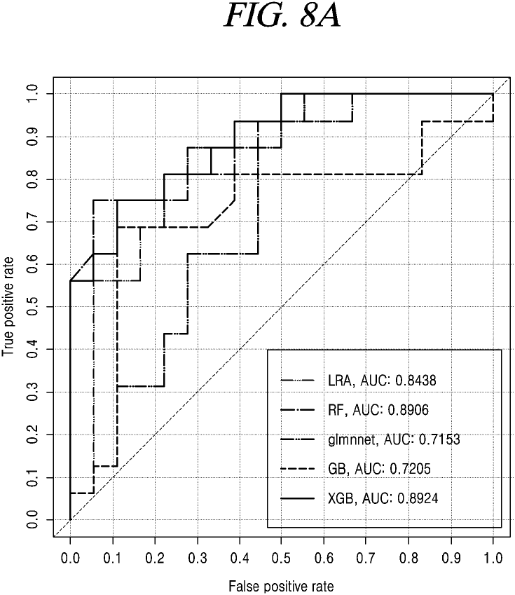

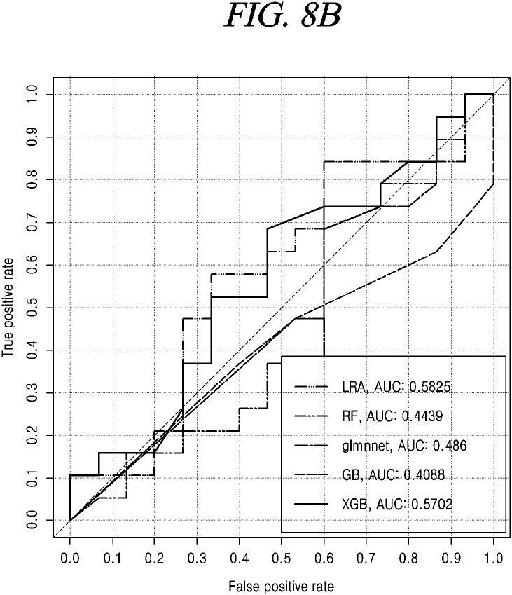

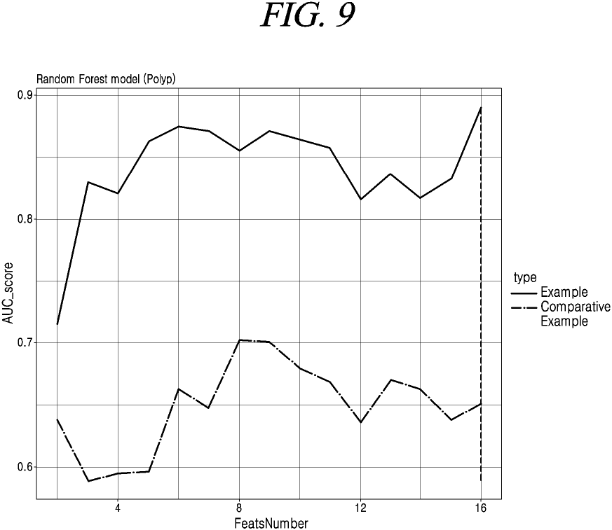

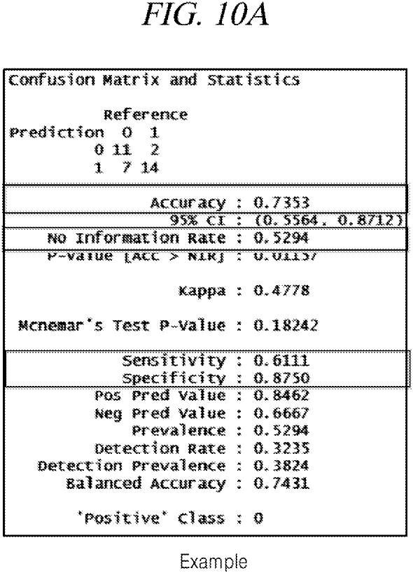

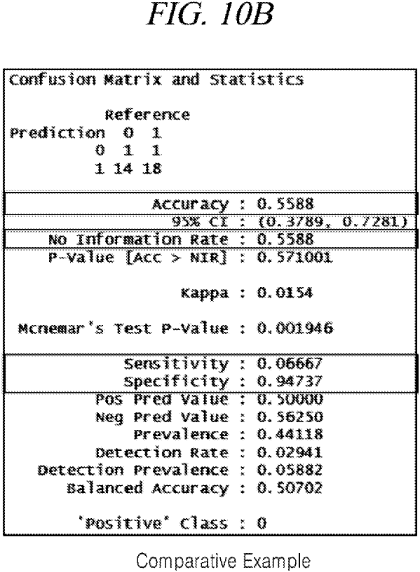

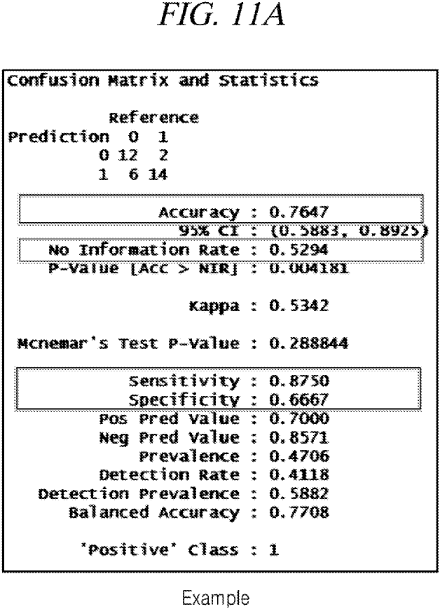

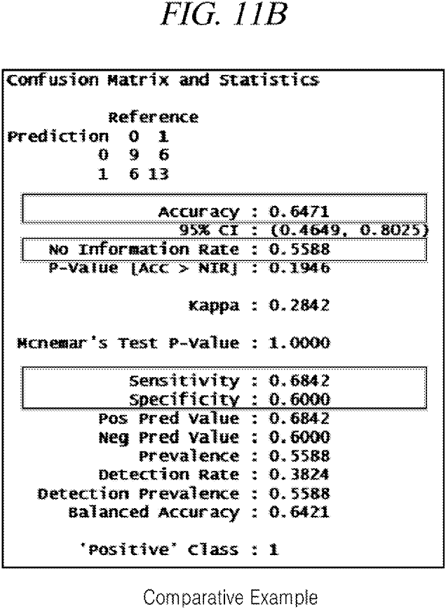

FIGS. 8A-8B are diagrams comparing machine learning models in performance according to the method of diagnosing the presence or absence of colon polyps of an example of the present disclosure and the method of Comparative Example, FIG. 9 is a diagram illustrating changes in performance of machine learning models depending on the number of features according to the method of diagnosing the presence or absence of colon polyps of an example of the present disclosure and the method of Comparative Example, FIGS. 10A-10B are diagrams comparing random forest models in performance according to the method of diagnosing the presence or absence of colon polyps of an example of the present disclosure and the method of Comparative Example, and FIGS. 11A-11B are diagrams comparing XGB models in performance according to the method of diagnosing the presence or absence of colon polyps of an example of the present disclosure and the method of Comparative Example.

FIGS. 8A-8B shows the Roc curve and AUC score of each machine learning model. As shown in FIGS. 8A-8B, when the machine learning models are trained with the microbial data of Example, it can be seen that all the machine learning models have higher performance than those of Comparative Example. Also, as shown in FIG. 9, the machine learning model of Example exhibits the highest performance when 16 features are selected.

FIGS. 10A-10B shows the accuracy, sensitivity and specificity of the random forest model trained with the microbial data of Example and the random forest model trained with the microbial data of Comparative Example, and FIGS. 11A-11B shows the accuracy, sensitivity and specificity of the XGB model trained with the microbial data of Example and the XGB model trained with the microbial data of Comparative Example.

Herein, the term “No Information Rate” refers to the accuracy of batch prediction for a test set as one group (disease or normal). For example, if a test set includes a disease group of 6 members and a test group of 4 members, the No Information Rate is 0.6 when prediction is made only for the disease group as the test set.

As shown in FIGS. 10A-10B and FIGS. 11A-11B, it can be seen that the machine learning model trained with the microbial data of Example has higher accuracy, sensitivity and specificity than the machine learning model trained with the microbial data of Comparative Example.

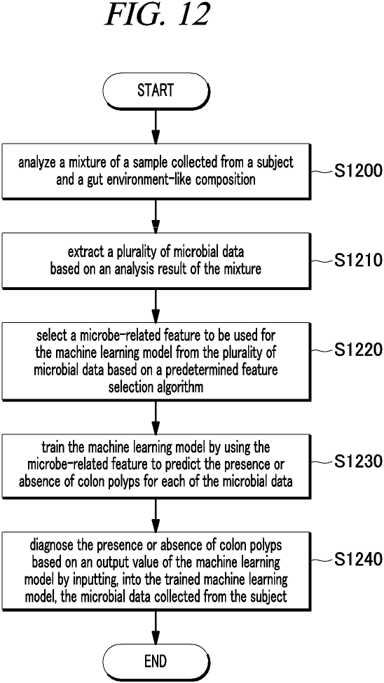

FIG. 12 is a flowchart illustrating a method of diagnosing the presence or absence of colon polyps according to an example of the present disclosure. The method of diagnosing the presence or absence of colon polyps according to the example illustrated in FIG. 12 includes the processes time-sequentially performed by the diagnostic apparatus illustrated in FIG. 1. Therefore, the above descriptions of the processes may also be applied to the method of diagnosing the presence or absence of colon polyps according to the example illustrated in FIG. 12, even though they are omitted hereinafter.

Referring to FIG. 12, a mixture of a sample collected from a subject and a gut environment-like composition may be analyzed in a process S1200.

In a process S1210, a plurality of microbial data may be extracted based on an analysis result of the mixture.

In a process S 1220, a microbe-related feature to be used for a machine learning model may be selected from the plurality of microbial data based on a predetermined feature selection algorithm.

In a process S1230, the machine learning model may be trained with the microbe-related feature.

In a process S1240, the machine learning model may be trained with the microbe-related feature.

The presence or absence of colon polyps can be diagnosed by inputting microbial data collected from a test subject into the trained machine learning model.

The method of diagnosing the presence or absence of colon polyps illustrated in FIG. 12 can be embodied in a storage medium including instruction codes executable by a computer such as a program module executed by the computer. A computer-readable medium can be any usable medium which can be accessed by the computer and includes all volatile/non-volatile and removable/non-removable media. Further, the computer-readable medium may include all computer storage media. The computer storage media include all volatile/non-volatile and removable/non-removable media embodied by a certain method or technology for storing information such as computer-readable instruction code, a data structure, a program module or other data.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

What is claimed is:

1. A method of diagnosing the presence or absence of colon polyps by using a machine learning model, which is performed by a diagnostic apparatus, comprising:
analyzing a mixture of a sample collected from a subject and a gut environment-like composition;
extracting a plurality of microbial data based on an analysis result of the mixture;
selecting a microbe-related feature to be used for the machine learning model from the plurality of microbial data based on a predetermined feature selection algorithm;
training the machine learning model by using the microbe-related feature to predict the presence or absence of colon polyps for each of the microbial data; and
diagnosing the presence or absence of colon polyps based on an output value of the machine learning model by inputting, into the trained machine learning model, the microbial data extracted based on the analysis result of the mixture of the sample collected from the subject and the gut environment-like composition,
wherein the microbe-related feature includes the content of at least one kind of microbes selected from families belonging to the order Oscillospirales, the order Burkholderiales, the order Saccharimonadales, the order Lactobacillales, the order Bacteroidales, the order Clostridiales, the order Erysipelotrichales, the order Bacteroidales and the order Lachnospirales.

2. The method of diagnosing the presence or absence of colon polyps of claim 1, wherein number of features to be used for the machine learning model is 6 to 16.

3. The method of diagnosing the presence or absence of colon polyps of claim 1,
wherein the analyzing a mixture includes:,
culturing the mixture in an anaerobic chamber for 18 hours to 24 hours under anaerobic conditions for 18 hours to 24 hours; and
analyzing, by the diagnostic apparatus, a culture in which the mixture has been cultured.

4. The method of diagnosing the presence or absence of colon polyps of claim 3,
wherein the analyzing a culture includes:
analyzing a supernatant and a precipitate obtained by centrifugation of the culture.

5. The method of diagnosing the presence or absence of colon polyps of claim 3,
wherein the microbial data includes at least one of the content, concentration and kind of substance contained in the culture, and a change in kind, concentration, content or diversity of bacteria included in microbiota, and
the substance contained in the culture includes at least one of endotoxins, hydrogen sulfides, short-chain fatty acids (SCFAs) and microbiota-derived metabolites.

6. The method of diagnosing the presence or absence of colon polyps of claim 1,
wherein the feature selection algorithm includes at least one of a Boruta algorithm and a recursive feature elimination (RFE) algorithm.

7. The method of diagnosing the presence or absence of colon polyps of claim 1,
wherein the machine learning model includes at least one of a logistic regression model, a glmnet model, a random forest model, a gradient boosting model and an extreme gradient boost (XGB) model.

8. The method of diagnosing the presence or absence of colon polyps of claim 1,
wherein the microbe-related feature includes the content of at least one kind of microbes selected from genera belonging to the family Oscillospiraceae, the family Streptococcaceae, the family Enterococcaceae, the family Marinifilaceae, the family Lactobacillaceae, the family Clostridiaceae, the family Leuconostocaceae, the family Erysipelatoclostridiaceae and the family Lachnospiraceae.

9. The method of diagnosing the presence or absence of colon polyps of claim 1,
wherein the microbe-related feature includes the content of at least one kind of microbes selected from species belonging to the genus Enterococcus, the genus Odoribacter, the genus Streptococcus, the genus Lactobacillus, the genus Clostridium sensu stricto, the genus leuconostoc, the genus Erysipelatoclostridium and the genus Eisenbergiella.

10. An apparatus of diagnosing the presence or absence of colon polyps by using a machine learning model, comprising:
a microbial data extraction unit that extracts a plurality of microbial data based on an analysis result of a mixture of a gut-derived substance collected from a subject and a gut environment-like composition;
a feature selection unit that selects a microbe-related feature to be used for the machine learning model from the plurality of microbial data based on a predetermined feature selection algorithm;
a training unit that trains the machine learning model by using the microbe-related feature to predict the presence or absence of colon polyps for each of the microbial data; and
a diagnosis unit that diagnoses colon polyps based on the presence or absence of colon polyps, which is an output value of the machine learning model, by inputting, into the trained machine learning model, the microbial data extracted based on the analysis result of the mixture of the gut-derived substance collected from the subject and the gut environment-like composition,
wherein the microbe-related feature includes the content of at least one kind of microbes selected from families belonging to the order Oscillospirales, the order Burkholderiales, the order Saccharimonadales, the order Lactobacillales, the order Bacteroidales, the order Clostridiales, the order Erysipelotrichales, the order Bacteroidales and the order Lachnospirales.

11. The apparatus of diagnosing the presence or absence of colon polyps of claim 10, wherein number of features to be used for the machine learning model is 6 to 16.

12. The apparatus of diagnosing the presence or absence of colon polyps of claim 10,
wherein the microbial data includes at least one of the content, concentration and kind of substance contained in the culture wherein the mixture is cultured in an anaerobic chamber for 18 hours to 24 hours under anaerobic conditions for 18 hours to 24 hours, and a change in kind, concentration, content or diversity of bacteria included in microbiota, and
the substance contained in the culture includes at least one of endotoxins, hydrogen sulfides, short-chain fatty acids (SCFAs) and microbiota-derived metabolites.

13. The apparatus of diagnosing the presence or absence of colon polyps of claim 10,
wherein the feature selection algorithm includes at least one of a Boruta algorithm and a recursive feature elimination (RFE) algorithm.

14. The apparatus of diagnosing the presence or absence of colon polyps of claim 10,
wherein the machine learning model includes at least one of a logistic regression model, a glmnet model, a random forest model, a gradient boosting model and an extreme gradient boost (XGB) model.

15. The apparatus of diagnosing the presence or absence of colon polyps of claim 10,
wherein the microbe-related feature includes the content of at least one kind of microbes selected from genera belonging to the family Oscillospiraceae, the family Streptococcaceae, the family Enterococcaceae, the family Marinifilaceae, the family Lactobacillaceae, the family Clostridiaceae, the family Leuconostocaceae, the family Erysipelatoclostridiaceae and the family Lachnospiraceae.

16. The apparatus of diagnosing the presence or absence of colon polyps of claim 10,
wherein the microbe-related feature includes the content of at least one kind of microbes selected from species belonging to the genus Enterococcus, the genus Odoribacter, the genus Streptococcus, the genus Lactobacillus, the genus Clostridium sensu stricto, the genus leuconostoc, the genus Erysipelatoclostridium and the genus Eisenbergiella.
